(19) United States
(12) Patent Application Publication
(10) Pub. No.: US 2026/0162815 A1
(43) Pub. Date: Jun. 11, 2026

(54) COMPUTER-IMPLEMENTED METHOD FOR FALL ASSESSMENT, AND ACTUATION, IMPLEMENTING TRAINED MACHINE LEARNING MODEL

(71) Applicant: AUTOLIV DEVELOPMENT AB, Vargarda (SE)

(72) Inventor: Jawwad AHMED, Vargarda (SE)

(21) Appl. No.: 18/704,344

(22) PCT Filed: Oct. 28, 2022
(86) PCT No.: PCT/EP2022/080177

§ 371 Date: Apr. 24, 2024

(30) Foreign Application Priority Data
Oct. 29, 2021 (SE) 2151329-6

(51) Int. Cl. G16H 40/63

(52) U.S. Cl. CPC G16H 40/63

(57) ABSTRACT

A computer-implemented method for fall assessment, for a fall assessment environment, wherein the computer-implemented method comprises implementing a trained machine learning model, comprising an action plan, and actuation in accordance to the action plan. The action plan has been generated reflecting the training of the machine learning model. The actuation, in accordance to the action plan, comprises protective measures, and reflects fall assessment, in relation to a user, wherein the fall assessment comprises fall detection and fall characterization of a detected fall. The computer-implemented method comprises continuously obtaining of data, and continuously processing of data, where the continuously obtained data comprise data detected by sensors worn by the user.

### DESCRIPTION OF THE DISCLOSURE

The present disclosure relates to an improved computer-implemented method for fall assessment, a computer program comprising computer readable instructions for applying the computer-implemented method, a computer program comprising computer readable instructions for a trained machine learning model, a computer program comprising computer readable instructions for a “fall characterizing machine learning unit”, computer readable mediums comprising the computer programs, as well as, a control unit arrangement, a device, and a system, for user fall assessment.

Falls are one leading cause of human injury and injury-related deaths. Fall detections and fall predictions are together with machine learning approaches of increased usefulness. Machine learning approaches are, for example, used today to distinguish fall and non-fall activities. At a high level, these approaches may include approaches involving vision-based arrangements which in real-time can classify falls based on live feed video. Vision-based arrangements may, in some instances, be less practical, e.g. in an outside environment, and may also come with potentially privacy issues. Further, there is also machine learning approaches focusing on more pervasive solutions being based on wearable device sensors which are non-intrusive and pervasive. In this area, see for example “A Cascade-Classifier Approach for Fall Detection, Putra et al, MOBIHEALTH 2015, Oct. 14-16, London, Great Britain, January 2015, DOI:10.4108/eai.14-10-2015.2261619” that proposes a cascade-classifier approach for this. Other examples include the M. Musci, D. D. Martini, N. Blago, T. Facchinetti, and M. Piastra, “Fall Detection using Recurrent Neural Networks,” p. 7 as well was the F. J. González-Cañete and E. Casilari, “A Feasibility Study of the Use of Smartwatches in Wearable Fall Detection Systems,” Sensors, vol. 21, no. 6, p. 2254, March 2021, doi: 10.3390/s21062254.

WO21050966 discloses systems and methods for predicting and preventing impending falls of a resident of a facility (e.g., a hospital, an assisted living facility, or a home) using a sensor. The system 100 generally can also be used to aid in preventing the falling of a resident and microphones may be used to determine information about type of fall, degree of severity of the fall.

The falls may for example occur for a person walking in a home environment, or outside the home. In this context, falls also comprise falls occurring when riding a bicycle, scooter, motorcycle and the like, for example due to skidding, slipping, losing control of vehicle, crash with another vehicle or object etc. Falls related to a running vehicle are often more abrupt, and require a faster handling than a fall occurring for a walking person that for example trips or slips.

However, there is still room for improvements in this area. In particular, regarding the requirements of improved methods for fall assessment.

This is achieved by means of a computer-implemented method for fall assessment, for a fall assessment environment, wherein the computer-implemented method comprises implementing a trained machine learning model, comprising an action plan, and actuation in accordance to the action plan. The action plan has been generated reflecting the training of the machine learning model. The actuation, in accordance to the action plan, comprises protective measures, and reflects fall assessment, in relation to a user, where the fall assessment comprises fall detection and fall characterization of a detected fall. The computer-implemented method comprises continuously obtaining of data, and continuously processing of data, where the continuously obtained data comprise data detected by sensors worn by the user.

This means that customized physical protection of the user can be provided using the generated action plan depending on the contextual scenario, for example anticipating the time before impact and which body area is going to have most impact after hitting (head, elbows, hips, sides) etc., and then triggering the physical protection accordingly.

According to some aspects, the protective measures includes physical measures which in turn include inflating an airbag.

This provides a quickly deployable type of protection that can be fitted at suitable positions. Said actuations may include, e.g. a mechanical actuation of a portable airbag after, for example, detection of a fall incident

According to some aspects, the actuation, in accordance to the action plan, comprises measures which in turn comprises at least one of preventive measures and alarming measures.

Alarming measures are suitable in case where time to impact is too short to do any protective measures, for example if the fall event is detected too late.

This means that the actuation comprises measures, such as, protective measures and/or alarming measures, wherein the protective measures includes physical measures such as inflating an airbag as soon as its detected that a person is in a dangerous position vulnerable to a fall such as a pre-fall situation.

According to some aspects, the computer-implemented method comprises continuous creation of information on basis of the obtained data and the processed data, wherein the computer-implemented method comprises continuous communication of the information. The computer-implemented method comprises fall assessment, in relation to a user, wherein the fall assessment comprises fall detection, and fall characterization, based on continuing data and on the continuing information.

According to some aspects, in accordance with the computer-implemented method for fall assessment, as described herein, for a fall assessment environment. T. assessment environment comprises the trained machine learning model being trained in fall assessment, the user, sensors detecting data from fall of user, and means for continuously obtaining data. The fall assessment environment further comprises means for processing data, means for creating the information on basis of obtained data and processed data, means for communicating the information, and means for the actuation in accordance to the action plan and based on continuing data and on the continuing information.

The trained machine learning model, being trained in fall assessment, continuously enables, during its implementation in the computer-implemented method for fall assessment: the continuous obtaining of data and the continuous processing of data,
the continuous creation, and the continuous communication, of the information,
the fall assessment, in relation to the user, comprising the fall detection and the fall characterization, and
the actuation in accordance to the action plan and reflecting the fall assessment.

According to some aspects, the computer-implemented method according to the present disclosure, as described herein, is disclosed, wherein the trained machine learning model, during its implementation in the computer-implemented method for fall assessment, continuously enables estimation of confidence in any predictions in the computer-implemented method. The computer-implemented method comprises estimation of confidence in any predictions of fall characterization, if the confidence in the prediction/s is/are high, actuation selection in accordance to the action plan and the prediction/s. If the confidence in the prediction/s is/are not high, default actuation in accordance to the action plan.

As an example, there may be a high confidence that a fall has occurred or is going to occur but still low confidence in the characterization of that fall.

According to some aspects, the computer-implemented method according to the present disclosure, as described herein, is disclosed, wherein the computer-implemented method comprises prediction of severity of the detected fall.

This may provide input for which protective and preventive measures that are to be activated, and to which degree.

According to some aspects, the computer-implemented method according to the present disclosure, as described herein, is disclosed, wherein the trained machine learning model, during its implementation in the computer-implemented method for fall assessment, continuously enables estimation of time to impact of the detected fall. The computer-implemented method comprises estimation of time to impact of the detected fall.

This means that factors such as for example fall characterization, fall severity and time-to-impact are computed and taken into account for the action plan for actuation. This may provide input for which protective and preventive measures that are to be activated, and to which degree.

The present disclosure also relates to computer programs, control units, devices, systems and pieces of garment that are associated with the above advantages.

Accordingly, it is here appreciated that the computer-implemented methods, the computer programs and the computer readable mediums, all as described herein, and in accordance with the present disclosure, may be realized in hardware, such as, the control unit arrangements and the devices, all as described herein, as well as, in the systems, as described herein. The hardware such as, the control unit arrangements and the devices, all as described herein, as well as, the systems, as described herein, are then arranged to perform the computer-implemented methods, and the computer programs, whereby the same advantages and effects are obtained as discussed for the computer-implemented methods herein.

### BRIEF DESCRIPTION OF DRAWINGS

The present disclosure will now be described more in detail with reference to the appended drawings, where:

FIG. 1 schematically illustrates a walking user wearing sensors;

FIG. 2 schematically illustrates a user who rides a motor bike and wears sensors;

FIG. 3 illustrates a schematic view over aspects of “Customized safety measure for fall incidents”, i.e. the action plan, and actuation in accordance to the action plan computer-implemented method for fall assessment, in accordance with the present disclosure;

FIG. 4 schematically illustrates a control unit; and

FIG. 5 shows an example computer program.

### DETAILED DESCRIPTION

Aspects of the present disclosure will now be described more fully hereinafter with reference to the accompanying drawings. The different arrangements, devices, systems, computer programs and methods disclosed herein can, however, be realized in many different forms and should not be construed as being limited to the aspects set forth herein. Like numbers in the drawings refer to like elements throughout.

The terminology used herein is for describing aspects of the disclosure only and is not intended to limit the invention. As used herein, the singular forms “a”, “an” and “the” are intended to include the plural forms as well, unless the context clearly indicates otherwise.

With reference to FIG. 1, there is a user 1 wearing at least one sensor device 2, 3, here one wrist sensor device 2 and one waist sensor device 3. In this case, the user 1 is walking.

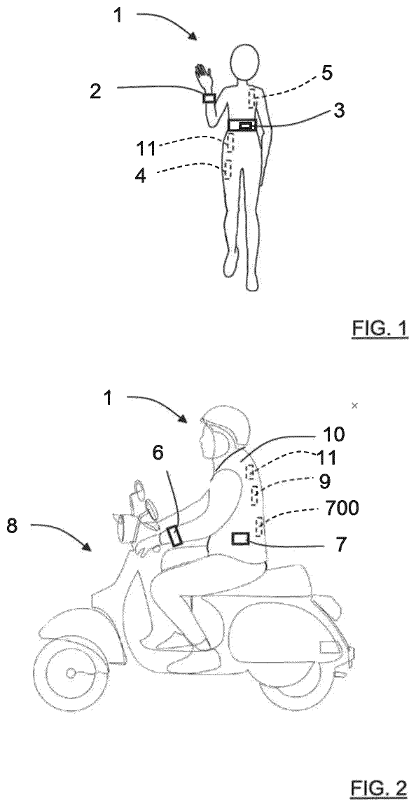

With reference also to FIG. 2, there is a user 1 wearing at least one sensor device, here one wrist sensor device 6 and one vest sensor device 7 comprised in a protection garment such as a protection vest 10. In this case, the user 1 is riding a motor bike 8.

The present disclosure is applicable for users that are walking or travelling on, or in, any kind of vehicle such as a bike, a motor bike 8, a car etc.

The obtained data, in accordance with present disclosure, comprise data obtained from the sensors 2, 3, 6, 7 and may also comprise data obtained from other sources. The data obtained from sensors 2, 3, 6, 7 are typically collected from, i.e. obtained from, sensors, e.g. motion sensors, comprised in, e.g. mounted on, wearable devices, for example 3d sensors, such as accelerometres to measure the acceleration in 3 dimensions, as well as, gyroscope to measure the rotational speed in 3 dimensions from a person 1, i.e. user, in accordance with present disclosure, wearing such a wearable device. The wearable devices 2, 3, 6, 7 may suitably also be edge devices. The obtained data may also be measurements from other sensors such as a magnetometer. A magnetometer is a device that measures magnetic field. Further, the obtained data may also comprise measurements of strength, or relative change, of a magnetic field at a particular location.

The obtained data may comprise data obtained from other sources than sensors. The obtained data may refer to current, and past historical, data. This may include geographical positioning information of the user from GPS system as well as weather information. Further information sources may include the static or semi-static information such as user physical profile e.g., age, height, BMI, any physical disabilities and also any relevant medical information such as vulnerability to falls retracted from the historical data etc., among other conditions. Such personal information of a user can be used in the computer-implemented method for fall assessment, as described herein, to tweak the fall severity computation for that specific user.

According to some aspects, the user 1 wears means 11 for providing a position of the user 1 and communicating said position. Such means 11 can comprise any type of suitable positioning system, such as for example GPS or GNSS (Global Navigation Satellite Systems) , and any type of wireless communication system. Said means can be comprised in a vest 11 or any suitable type of garment or wearable device.

The present disclosure relates to a computer-implemented method for fall assessment, for a fall assessment environment, wherein the computer-implemented method comprises implementing a trained machine learning model, comprising an action plan, and actuation in accordance to the action plan, where the action plan has been generated reflecting the training of the machine learning model.

The actuation, in accordance to the action plan, comprises protective measures, and reflects fall assessment, in relation to a user 1, wherein the fall assessment comprises fall detection and fall characterization of a detected fall. The computer-implemented method comprises continuously obtaining of data, and continuously processing of data, where the continuously obtained data comprise data detected by sensors 2, 3, 6, 7 worn by the user 1.

This means that customized physical protection of the user 1 can be provided using the generated action plan depending on the contextual scenario, for example anticipating the time before impact and which body area is going to have most impact after hitting (head, elbows, hips, sides) etc., and then triggering the physical protection accordingly.

According to some aspects, the protective measures includes physical measures which in turn include inflating an airbag.

This provides a quickly deployable type of protection that can be fitted at suitable positions. Furthermore, this means that said actuations may include, e.g. a mechanical actuation of a portable airbag 4, 5; 9 after, for example, detection of a fall incident. The safety capabilities of the portable airbag 4, 5; 9, and properties such as volume, inflation capacity and inflation rate of the airbag 4, 5; 9, is based on the action plan, and on the inducement in accordance to the action plan. The inducement comprises actuations, and preventive measures, and correlates to, and/or reflects, fall assessment in relation to a user, wherein the fall assessment comprises fall detection, and computation of FRP, for that specific user.

According to some aspects, the actuation, in accordance to the action plan, comprises measures which in turn comprises at least one of preventive measures and alarming measures.

Alarming measures are suitable in case where time to impact is too short to do any protective measures, for example if the fall event is detected too late.

According to some aspects, the actuation comprises measures, such as, protective measures and/or alarming measures, wherein the alarming measures includes alarming the user of the present computer-implemented method for fall assessment, as described herein, when user is in a dangerous situation, for example, before a pre-fall situation and where the user may risk being injured or hurt. Thus, the user may then be able to quickly do some corrective measure and/or, e.g., any walk posture corrections to avoid a potential injury. The alarming measures may have the nature of visual warnings such as on a small screen or using a bulb, audible alerts and/or haptic feedback patterns, to alert the user of a potentially dangerous upcoming situation.

In this context, physical measures are a part of protective measures that in turn relate to something that protects a person who falls. Preventive measures are referring to measures that prevent a fall such as an alarm when there is a high risk that a fall might occur. In a motorcycle driver case it may a warning issued in dependence of detected slippery/icy road conditions.

According to some aspects, preventive measures include warning signals and protective measures include inflation of one or more protection airbags 4, 5; 9 as illustrated in FIG. 1 and FIG. 2. As shown in FIG. 2, for a person or user 1 riding a motor bike 8 or similar, sensors 6, 7, control units 700 and/or protection airbags 9 can all be comprised in a protection garment such as a protection vest 10.

Further, the preventive measures refer to counter measures to avoid a fall incident in the first place before the incident takes place. The preventive measures include, but are not limited to, actual alerts, for example visual, audible, and or haptic alerts, etc., to the user/s if a posture is detected that is a posture in a dangerous position e.g. being close to fall (but where a point of no return has still not been reached).

Further, the preventive measures may also include, other physical preventive measures such as increased protective clothing and/or protective gear for the user/s, and/or may even include increased monitoring of the user/s. Further, these preventive measures are also based on the fall assessment wherein the fall assessment comprises fall detection, and computation of FRP for the specific user, and may also, as described herein, comprise the computation of FRP in conjunction with geo positioning of a GPS, e.g. of FRP in conjunction with geo positioning of a user currently utilizing GPS.

The computer-implemented method for fall assessment, as described herein, comprises the actuation in accordance to the action plan, wherein the action plan has been generated reflecting the training of the machine learning model, and wherein the actuation reflects fall assessment, in relation to a user, wherein the fall assessment comprises fall detection and fall characterization.

The action plan has been generated reflecting the training of the machine learning model, wherein generation of the action plan is based on outputs of different trained machine learning models which predict traits such as the type of fall that is going to occur (if fall is predicted to occur) as well as the severity of the fall based on factors such as type of fall and acceleration/speed of hitting the ground/impact.

The fall detection is a first step to detect a fall and it triggers other mechanisms to generate the action plan, and also do the preventive and protective measures. The fall detection is performed in real-time using the trained machine learning model on the data collected, i.e. the data obtained from user, e.g. being trainers, instrumented with the 3D sensors. A trained fall detection machine learning model will generate a signal with a certain lead time if a fall is upcoming based on the current, and past, state data of a user instrumented with, and using, the 3d sensors.

The fall characterization refers to identification features such as fall type characterization such as, for example, frontal falls, sideway falls, backward falls such as from a slip among others. The method for this could be based on a data-driven approach such as machine learning or based on an analytical method based on domain knowledge.

Another important feature, of the fall characterization, is a classification method for severity of the fall correlating to the probability of severe injury from the fall. This could be based on data collected in real-time from the sensors but also historical data for the user based on aspects such as medical history, sickness condition and age etc.

According to some aspects, the computer-implemented method comprises continuous creation of information on basis of the obtained data and the processed data, wherein the computer-implemented method comprises continuous communication of the information. The computer-implemented method comprises fall assessment, in relation to a user, wherein the fall assessment comprises fall detection, and fall characterization, based on continuing data and on the continuing information.

The computer-implemented method, as described herein, comprises fall assessment, in relation to a user, wherein the fall assessment comprises fall detection, and fall characterization, based on continuing data and on the continuing information.

The computer-implemented method, as described herein, comprises the fall detection, based on continuing data and on the continuing information, wherein a fall detection machine learning model is trained on the real-time data collected from 3D sensors.

Moreover, a fall characterization computer program, comprising. instructions for the “fall characterization machine learning unit”, is employed.

The computer-implemented method, as described herein, further comprises the fall characterization, based on continuing data and on the continuing information. The fall characterization is only triggered if a fall is detected in the first place by the fall detection machine learning model. The intention with the computer-implemented method, comprising the fall characterization, is to detect and/or predict the type of a fall and/or the nature of a fall.

Further, according to some aspects, a fall severity computer program, comprising instructions for a “fall severity prediction machine learning module”, is also employed in connection with the fall type prediction. The fall severity computer program detects the severity of fall or the probability for a severe injury from a specific fall, e.g. a specific fall type. This fall severity computer program can take as input, i.e. obtain, the real-time data streaming from 3D sensors, as well as, historical data from a user, and its user profile, and also other information to estimate the sever injury probability from the fall impact for a specific user. If both these traits are able to be predicted with high confidence and/or probability then this will enable the generation of a customized action plan according to the needs of the specific user in that fall scenario if a fall is detected in the first place.

According to some aspects, the fall assessment environment comprises the trained machine learning model being trained in fall assessment, the user 1, sensors 2, 3, 6, 7 detecting data from fall of user, means for continuously obtaining data, and means 700 for processing data. The fall assessment environment further comprises means for creating the information on basis of obtained data and processed data, means for communicating the information, and means for the actuation in accordance to the action plan and based on continuing data and on the continuing information. The trained machine learning model, during its implementation in the computer-implemented method for fall assessment, continuously enables
the continuous obtaining of data and the continuous processing of data,
the continuous creation, and the continuous communication, of the information,
the fall assessment, in relation to the user 1, comprising the fall detection and the fall characterization, and
the actuation in accordance to the action plan and reflecting the fall assessment.

The computer-implemented method, as described herein, further comprises that the “fall characterizing machine learning unit” comprises a “fall type prediction machine learning module”, wherein the “fall type prediction machine learning module”, during said implementation of the trained machine learning model and when a fall is detected, enables:
a prediction of fall type of the detected fall; and
that the computer-implemented method for fall assessment comprises the fall characterization of a detected fall, wherein the fall characterization comprises a prediction of fall type, and
the actuation: in accordance to the action plan and the predicted fall type.

Further, the prediction of the type of fall together with the associated severity with high confidence will enable the proposed computer-implemented method for fall assessment, as described herein, following the fall detection and creating an optimized actuation plan customized for the specific user under that specific fall incident dynamics, the optimized actuation plan comprising actuation such as, for example, actuation of a portable airbag in a specific direction based on the fall type, as well as, based on other factors such as depending on the severity of the fall and adjusting parameters of actuation such as inflation speed and/or capacity or triggering of multiple portable airbags depending on the fall type and the severity for specific the specific user.

This is driven by the action plan. Time to impact provides further feedback to assess if there is sufficient time available to do a certain actuation before an impact occurs and an injury is potentially sustained. A main objective of the computer-implemented method for fall assessment, as described herein, will be to maximize the protection of the user from serious injury if a fall incident occurs depending on the user profile, medical condition, fall dynamics and environmental conditions.

The computer-implemented method further comprises the fall characterization, wherein the fall characterization comprises a prediction of fall type of a detected fall.

According to some aspects, the trained machine learning model, during its implementation in the computer-implemented method for fall assessment, continuously enables estimation of confidence in any predictions of fall characterization in the computer-implemented method. The computer-implemented method comprises estimation of confidence in any predictions, if the confidence in the prediction/s is/are high, actuation selection in accordance to the action plan and the prediction/s, and, if the confidence in the prediction/s is/are not high, default actuation in accordance to the action plan.

Fall characterization may for example include type of fall and/or severity. As an example, there may be a high confidence that a fall has occurred or is going to occur but still low confidence in the characterization of that fall.

According to some aspects, the computer-implemented method comprises prediction of severity of the detected fall.

This may provide input for which protective and preventive measures that are to be activated, and to which degree.

According to some aspects, the trained machine learning model, during its implementation in the computer-implemented method for fall assessment, continuously enables estimation of time to impact of the detected fall. The computer-implemented method further comprises estimation of time to impact of the detected fall.

This means that factors such as for example fall characterization, fall severity and time-to-impact are computed and taken into account for the action plan for actuation. This may provide input for which protective and preventive measures that are to be activated, and to which degree.

In further aspects of the present disclosure, as described herein, the fall characterization comprises the prediction of fall type of the detected fall, and the fall characterization further comprises estimating confidence in any predictions and/or predicting severity of the detected fall.

According to some aspects, the fall characterization comprises a prediction of fall type, where the actuation is performed in accordance to the action plan and the predicted fall type.

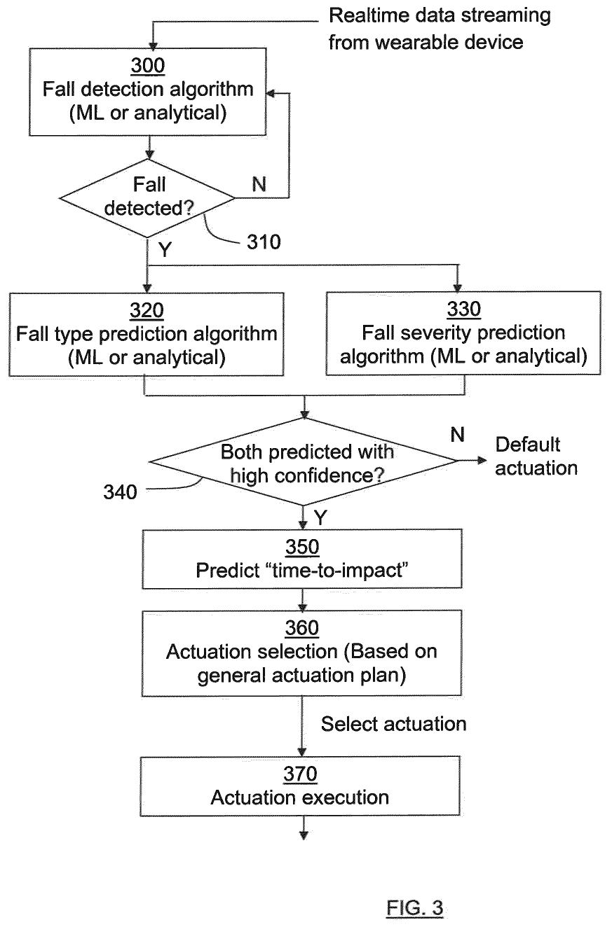

A proposed solution, e.g. in accordance with the computer-implemented method for fall assessment, as described herein, is depicted in FIG. 3. FIG. 3 relates to aspects of “Customized safety measure for fall incidents”, i.e. the action plan, and actuation in accordance to the action plan of the computer-implemented method for fall assessment, in accordance with the present disclosure. In the FIG. 3 it can be seen that data is streaming in real-time from sensors 2, 3, 6, 7 mounted on wearable devices which could be edge, or IoT, devices. This data is used by the trained fall detection algorithm, i.e. the trained fall detection computer program, in the trained fall detection machine learning model, to detect a fall, see “Fall Detection ML Algorithm (ML or Analytical)” 300, “Fall detected?” 310 in FIG. 33, “Y” means yes, and “N” means no.

The fall detection algorithm, i.e. the fall detection computer program, and the fall detection machine learning model, are trained by the training data which are collected by the user 1, e.g. being trainers or being other users, such as motorcycle and bicycle riders, wearing the device and collecting the relevant fall data to train the fall detection algorithm, i.e. the fall detection computer program, and the fall detection machine learning model. Data may also be either labeled at the time when the data is being collected, or labeled offline later using either a manual approach or an automated approach. If a fall is predicted by the fall detection algorithm, i.e. the fall detection computer program, then another algorithm, i.e. another computer program, is triggered namely the fall type prediction algorithm, i.e. the fall type prediction computer program, in the “fall type prediction machine learning module”, which predicts the type of fall which has been detected. The “fall type prediction algorithm (ML or Analytical)” 320 and “prediction of fall type of a detected fall” are comprised in the fall characterization, in accordance with the present disclosure, and the “fall characterizing machine learning unit” comprises the “fall type prediction machine learning module”, in accordance with the present disclosure, and as described herein. Exact fall type classification, i.e. fall type characterization, i.e. both comprised in the fall characterization, in accordance with the present disclosure, will be dependent on a specific user case, for example, one way is to classify, i.e. characterize, the fall basing classification on the fall angle such as fall forward, fall backward, fall sideway (left/right) or fall on the spot without noticeable leaning towards any side or angle, or as slip based falls, and so forth.

In parallel a fall severity prediction algorithm 330, i.e. a fall severity prediction computer program, which may, or may not, be comprised in the “fall severity prediction machine learning module” is employed, and the fall severity prediction algorithm 330 may, or may not, be based on machine learning, see Fall Severity Prediction Algorithm (ML or Analytical) 330 in FIG. 3. This fall severity prediction algorithm 330, i.e. the fall severity prediction computer program, will predict and/or estimate the severity of fall based on the fall acceleration, angle and other parameters (such as position, slope). The fall severity prediction algorithm 330 is comprised in “fall severity prediction machine learning module”, in accordance with the present disclosure.

As indicated above, “fall detection” 300, “fall type prediction” and “fall severity prediction” 330 may or may not be based on ML. Non-ML based methods can for example be based on domain knowledge.

If both of these traits, i.e. “fall type prediction” and the “fall severity prediction”, is not predicted with high certainty (or confidence), see “Both predicted with high confidence?” 340, in FIG. 3, “Y” means yes, and “N” means no, and also “Fall detected?” in FIG. 3 then a predefined default actuation method, i.e. actuation, can be signaled to be triggered, see “Default Actuation” in FIG. 3. Otherwise, if both of the traits are triggered indeed with a high confidence then the time-to-impact is calculated, see “Predict “time-to-impact”” 350 in FIG. 3 which eventually leads to an actuation selection algorithm 360, i.e. an actuation selection computer program, which is extracted from an already generated action plan for actuation based on predicted and calculated traits such as fall type, fall severity and predicted time-to-impact. Finally, a selected actuation is signaled to be executed 370, where the selected actuation will be suitable for that specific user for that specific crash incident, i.e. fall incident.

According to some aspects, the computer-implemented method according to the present disclosure, as described herein, is disclosed, wherein the “fall characterizing machine learning unit” comprises a “fall severity prediction machine learning module”, wherein the “fall severity prediction machine learning module”, during said implementation of the trained machine learning model enables the prediction of severity of the detected fall.

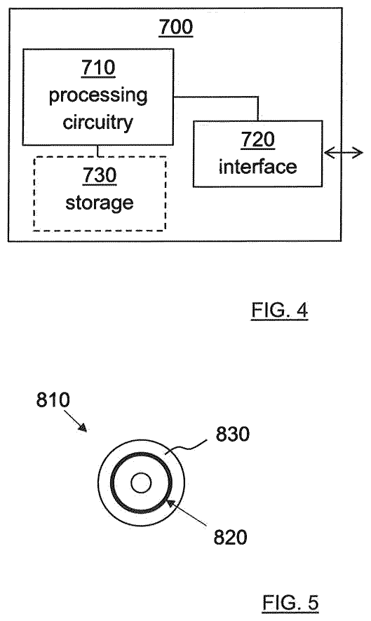

Further, with reference to FIG. 4 the present disclosure also relates to a control unit arrangement 700, for user fall assessment, adapted to control at least, enablement of: the implementation of the trained machine learning model, as described herein, and/or the actuation in accordance to an action plan of the trained machine learning model, as described herein.

FIG. 4 schematically illustrates, in terms of a number of functional units, the components of the control unit 700 according to an embodiment.

Processing circuitry 710 is provided using any combination of one or more of a suitable central processing unit (CPU), multiprocessor, microcontroller, digital signal processor (DSP), dedicated hardware accelerator, etc., capable of executing software instructions stored in a computer program product, e.g. in the form of a storage medium 730. The processing circuitry 710 may further be provided as at least one application specific integrated circuit (ASIC), or field programmable gate array (FPGA).

Particularly, the processing circuitry 710 is configured to cause the control unit 700 to perform a set of operations, or steps. These operations, or steps, were discussed above in connection to the various radar transceivers and methods. For example, the storage medium 730 may store the set of operations, and the processing circuitry 710 may be configured to retrieve the set of operations from the storage medium 730 to cause the control unit 700 to perform the set of operations. The set of operations may be provided as a set of executable instructions. Thus, the processing circuitry 710 is thereby arranged to execute methods and operations as herein disclosed.

The storage medium 730 may also comprise persistent storage, which, for example, can be any single one or combination of magnetic memory, optical memory, solid state memory or even remotely mounted memory.

The control unit 700 may further comprise a communications interface 720 for communications with at least one other unit. As such, the communications interface 720 may comprise one or more transmitters and receivers, comprising analogue and digital components and a suitable number of ports for wired or wireless communication.

The processing circuitry 710 is adapted to control the general operation of the control unit 700 e.g. by sending data and control signals to the external unit and the storage medium 730, by receiving data and reports from the external unit, and by retrieving data and instructions from the storage medium 730. Other components, as well as the related functionality, of the control unit 700 are omitted in order not to obscure the concepts presented herein.

It is to be appreciated that for all embodiments disclosed herein, FIG. 5 discloses a general representation of a computer program 810 comprising computer readable instructions 820 on a computer readable medium 830. The computer program may according to some aspects be regarded as a computer program product.

The present disclosure also relates to a computer program 810 comprising computer readable instructions 820 for applying the computer-implemented method, as described herein, and a computer readable medium 830 comprising said computer program 810.

The present disclosure also relates to a computer program 810 comprising computer readable instructions 820 for a trained machine learning model, as described herein, and a computer readable medium 830 comprising said computer program 810.

The present disclosure also relates to a computer program 810 comprising computer readable instructions 820 for a “fall characterizing machine learning unit”, as described herein, and a computer readable medium 830 comprising said computer program 810.

The present disclosure also relates to a device, e.g. a wearable device, for user fall assessment, for communication of information, for enabling implementation of a trained machine learning model, wherein the trained machine learning model is as described herein, and for enabling actuation in accordance to an action plan of the trained machine learning model. The action plan is generated using a fall characterizing machine learning unit, wherein the fall characterizing machine learning unit is trained in fall characterizing, where the fall assessment comprises fall detection.

The device comprises the trained machine learning model being trained in the user fall assessment and comprising the action plan, device sensor/s 2, 3, 6, 7 detecting data, means for continuously obtaining data, means 700 for processing data, means for creating the information on basis of obtained data and processed data, and means for communicating the information.

The device comprises the computer programs 810, and/or the computer readable mediums 830, all as described herein, for applying the computer-implemented method, as described herein, for the trained machine learning model, as described herein; and/or for the “fall characterizing machine learning unit”, as described herein.

According to some aspects, the device is at least partly in the form of a vest 10 or similar garment. The device can also be constituted by a control unit arrangement 700 and/or a bracelet or similar. In FIG. 1, a user 1 is shown hearing a sensor 2 that is comprised in a bracelet. Generally, the present disclosure relates to a 15 a piece of garment 10 comprising the control unit arrangement 700 as described herein, one or more sensors 2, 3, 6, 7 detecting data as described herein, and at least one airbag 4, 5, 9.

The present disclosure also relates to a system for user fall assessment, for communication of information, for enabling implementation of a trained machine learning model, wherein the trained machine learning model is as described herein, and for enabling actuation in accordance to an action plan of the trained machine learning model. The action plan is generated using a fall characterizing machine learning unit, wherein the fall characterizing machine learning unit is trained in fall characterizing, where the fall assessment comprises fall detection.

The system comprises the trained machine learning model being trained in the user fall assessment and comprising the action plan, sensor/s 2, 3, 6, 7 detecting data, means for continuously obtaining data, means 700 for processing data, means for creating the information on basis of obtained data and processed data, and means for communicating the information.

The system comprises the computer programs 810, and/or the computer readable mediums 830, all as described herein, for applying the computer-implemented method, as described herein, for the trained machine learning model, as described herein, and/or for the “fall characterizing machine learning unit”, as described herein.

According to some aspects, the present disclosure relates to a computer-implemented method for fall assessment, for a fall assessment environment, wherein the computer-implemented method comprises implementing a trained machine learning model, comprising a “fall characterizing machine learning unit” and an action plan, and actuation in accordance to the action plan, wherein the action plan has been generated reflecting the training of the machine learning model.

The actuation, in accordance to the action plan, comprises measures, such as, protective measures, preventive measures and/or alarming measures, and reflects fall assessment, in relation to a user, wherein the fall assessment comprises fall detection and fall characterization. The computer-implemented method comprises continuously obtaining data, and continuously processing of data, wherein the computer-implemented method comprises continuous creation of information on basis of the obtained data and the processed data, wherein the computer-implemented method comprises continuous communication of the information, wherein the computer-implemented method comprises fall assessment, in relation to a user, wherein the fall assessment comprises fall detection, and fall characterization, based on continuing data and on the continuing information.

The fall assessment environment comprises the trained machine learning model being trained in fall assessment, the user 1, sensors 2, 3, 6, 7 detecting data from fall of user 1, means for continuously obtaining data, means 700 for processing data, means for creating the information on basis of obtained data and processed data, means for communicating the information, and means for the actuation in accordance to the action plan and based on continuing data and on the continuing information.

The trained machine learning model, during its implementation in the computer-implemented method for fall assessment, continuously enables the continuous obtaining of data and the continuous processing of data, the continuous creation, and the continuous communication, of the information, the fall assessment, in relation to the user, comprising the fall detection and the fall characterization, and the actuation in accordance to the action plan and reflecting the fall assessment.

The “fall characterizing machine learning unit” comprises a “fall type prediction machine learning module”, wherein the “fall type prediction machine learning module”, during said implementation of the trained machine learning model and when a fall is detected, enables a prediction of fall type of the detected fall. The computer-implemented method for fall assessment comprises the fall characterization of a detected fall, wherein the fall characterization comprises a prediction of fall type, and the actuation in accordance to the action plan and the predicted fall type.

According to some aspects, there can be other sensors than the above 3D sensors, for example 1D/2D motion sensors as well as magnetometer, GPS, etc.

1. A computer-implemented method for fall assessment, for a fall assessment environment,
wherein the computer-implemented method comprises implementing a trained machine learning model, comprising an action plan, and actuation in accordance to the action plan,
wherein the action plan has been generated reflecting the training of the machine learning model,
wherein the actuation, in accordance to the action plan, comprises protective measures, and reflects fall assessment, in relation to a user, wherein the fall assessment comprises fall detection and fall characterization of a detected fall, and
wherein the computer-implemented method comprises continuously obtaining of data, and continuously processing of data, where the continuously obtained data comprise data detected by sensors worn by the user.

2. The computer-implemented method according to claim 1, wherein the protective measures includes physical measures which in turn include inflating an airbag.

3. The computer-implemented method according to claim 1, wherein the actuation, in accordance to the action plan, comprises measures which in turn comprises at least one of preventive measures and alarming measures.

4. The computer-implemented method according to claim 1,
wherein the computer-implemented method comprises continuous creation of information on basis of the obtained data and the processed data,
wherein the computer-implemented method comprises continuous communication of the information,
wherein the computer-implemented method comprises fall assessment, in relation to a user, and
wherein the fall assessment comprises fall detection, and fall characterization, based on continuing data and on the continuing information.

5. The computer-implemented method according to claim 1, wherein the fall assessment environment comprises:
the trained machine learning model being trained in fall assessment;
the user;
sensors detecting data from fall of user;
means for continuously obtaining data;
means for processing data;
means for creating the information on basis of obtained data and processed data;
means for communicating the information; and
means for the actuation in accordance to the action plan and based on continuing data and on the continuing information;
wherein the trained machine learning model, during its implementation in the computer-implemented method for fall assessment, continuously enables:
the continuous obtaining of data and the continuous processing of data;
the continuous creation, and the continuous communication, of the information;
the fall assessment, in relation to the user, comprising the fall detection and the fall characterization, and
the actuation in accordance to the action plan and reflecting the fall assessment.

6. The computer-implemented method according to claim 1, wherein the trained machine learning model, during its implementation in the computer-implemented method for fall assessment, continuously enables estimation of confidence in any predictions in the computer-implemented method, and
wherein the computer-implemented method comprises estimation of confidence in any predictions of fall characterization, if the confidence in the prediction/s is/are high, actuation selection in accordance to the action plan and the prediction/s, and,
if the confidence in the prediction/s is/are not high, default actuation in accordance to the action plan.

7. The computer-implemented method according to claim 2, wherein the computer-implemented method comprises prediction of severity of the detected fall.

8. The computer-implemented method according to claim 1, wherein the trained machine learning model, during its implementation in the computer-implemented method for fall assessment, continuously enables estimation of time to impact of the detected fall, and
wherein the computer-implemented method comprises estimation of time to impact of the detected fall.

9. The computer-implemented method according to claim 1, wherein the fall characterization comprises a prediction of fall type, where the actuation is performed in accordance to the action plan and the predicted fall type.

10. A computer program comprising computer readable instructions for applying the computer-implemented method according to claim 1, and a computer readable medium comprising said computer program.

11. A computer program comprising computer readable instructions for a trained machine learning model according to claim 1, and a computer readable medium comprising said computer program.

12. A control unit arrangement, for user fall assessment, adapted to control at least, enablement of:
the implementation of the trained machine learning model according to claim 1, and/or
the actuation in accordance to an action plan of the trained machine learning model.

13. A device, e.g. a wearable device, for user fall assessment, for communication of information, for enabling implementation of the trained machine learning model according to claim 1, and for enabling actuation in accordance to an action plan of the trained machine learning model;
wherein the action plan is generated using a fall characterizing machine learning unit,
wherein the fall characterizing machine learning unit is trained in fall characterizing,
wherein the fall assessment comprises fall detection,
wherein the device comprises:
the trained machine learning model being trained in the user fall assessment and comprising the action plan;
device sensor/s detecting data;
means for continuously obtaining data;
means for processing data;
means for creating the information on basis of obtained data and processed data; and
means for communicating the information;
wherein the device comprises a computer program comprising computer readable instructions for applying the computer-implemented method, and a computer readable medium comprising said computer program.

14-15. (canceled)

16. A device, e.g. a wearable device, for user fall assessment, for communication of information, for enabling implementation of the trained machine learning model according to claim 1, and for enabling actuation in accordance to an action plan of the trained machine learning model;
wherein the action plan is generated using a fall characterizing machine learning unit,
wherein the fall characterizing machine learning unit is trained in fall characterizing,
wherein the fall assessment comprises fall detection,
wherein the device comprises:
the trained machine learning model being trained in the user fall assessment and comprising the action plan;
device sensor/s detecting data;
means for continuously obtaining data;
means for processing data;
means for creating the information on basis of obtained data and processed data; and
means for communicating the information,
wherein the device comprises a computer program comprising computer readable instructions for the trained machine learning model, and a computer readable medium comprising said computer program.

17. A system for user fall assessment, for communication of information, for enabling implementation of the trained machine learning model according to claim 1, and for enabling actuation in accordance to an action plan of the trained machine learning model;
wherein the action plan is generated using a fall characterizing machine learning unit,
wherein the fall characterizing machine learning unit is trained in fall characterizing,
wherein the fall assessment comprises fall detection,
wherein the system comprises:
the trained machine learning model being trained in the user fall assessment and comprising the action plan;
sensor/s detecting data;
means for continuously obtaining data;
means for processing data;
means for creating the information on basis of obtained data and processed data; and
means for communicating the information,
wherein the system comprises a computer program comprising computer readable instructions for applying the computer-implemented method, and a computer readable medium comprising said computer program.

18. A system for user fall assessment, for communication of information, for enabling implementation of the trained machine learning model according to claim 1, and for enabling actuation in accordance to an action plan of the trained machine learning model;
wherein the action plan is generated using a fall characterizing machine learning unit,
wherein the fall characterizing machine learning unit is trained in fall characterizing,
wherein the fall assessment comprises fall detection,
wherein the system comprises:
the trained machine learning model being trained in the user fall assessment and comprising the action plan;
sensor/s detecting data;
means for continuously obtaining data;
means for processing data;
means for creating the information on basis of obtained data and processed data; and
means for communicating the information,
wherein the system comprises a computer program comprising computer readable instructions for the trained machine learning model, and a computer readable medium comprising said computer program.

19. A piece of garment comprising the control unit arrangement according to claim 12, one or more sensors detecting data, and at least one airbag.
